(19) United States
(12) Patent Application Publication
(10) Pub. No.: US 2024/0060105 A1
(43) Pub. Date: Feb. 22, 2024

(54) Improved Production of Melanin in Vibirio Natriegens

(71) Applicant: The Government of the United States of America, as represented by the Secretary of the Navy, Arlington, VA (US)

(72) Inventors: Zheng WANG, Burke, VA (US); Aaron SMITH, Alexandria, VA (US); Tanya TSCHIRHART, Odenton, MD (US); Jaimee COMPTON, Washington, DC (US)

(21) Appl. No.: 18/229,933

(22) Filed: Aug. 3, 2023

Related U.S. Application Data
(60) Provisional application No. 63/398,555, filed on Aug. 17, 2022.

(51) Int. Cl. C12P 17/18; C12N 1/20; C12N 15/74; C12N 9/02

(52) U.S. Cl. CPC C12P 17/182; C12N 1/20; C12N 15/74; C12N 9/0071; C12Y 114/18001; C12R 2001/63

(57) ABSTRACT

Improved yields of melanin are obtained by incubating, in an optimized media with a disodium tyrosine substrate, a culture of Vibrio natriegens expressing a heterologous tyrosinase gene at a temperature of about 30° C.

### CROSS-REFERENCE TO RELATED APPLICATIONS

This application claims the benefit of U.S. Provisional Patent Application No. 63/398,555 filed on Aug. 17, 2023, the entirety of which is incorporated herein by reference.

### FEDERALLY-SPONSORED RESEARCH AND DEVELOPMENT

The United States Government has ownership rights in this invention. Licensing inquiries may be directed to Office of Technology Transfer, US Naval Research Laboratory, Code 1004, Washington, DC 20375, USA; +1.202.767.7230; techtran@nrl.navy.mil, referencing NC 211,155.

### BACKGROUND

Melanins are macromolecules formed by oxidative polymerization of phenolic and/or indolic compounds. These black or brown pigments are hydrophobic, negatively charged, and ubiquitous in nature and impart a large variety of biological functions to organisms, including structure, coloration, free radical scavenging, radiation resistance, and thermoregulation. Inspired by the physicochemical, optoelectronic, self-assembling, and adhesive properties of natural melanin, a number of research groups have synthesized melanin nanoparticles for a broad range of applications, including protective coatings, functional films, environmental sensors, and energy storage devices. For example, melanin can be used in chemical protective materials, such as garments, as described in commonly-owned U.S. Pat. No. 11,162,212.

A process for production of melanin was described in Wang et al., “Melanin Produced by the Fast-Growing Marine Bacterium Vibrio natriegens through Heterologous Biosynthesis: Characterization and Application,” Applied and Environmental Microbiology, 2020, 86 (5), e02749-19 (hereinafter, “Wang et al.”, incorporated herein by reference for the purposes of disclosing techniques for obtaining melanin from cultures of Vibrio). Under the conditions described therein, melanin with yield were approximately 1 g per liter.

A need exists for improved yields in the production of melanin from Vibrio natriegens.

### BRIEF SUMMARY

In one embodiment, a method for producing melanin comprises incubating a culture of Vibrio natriegens expressing a tyrosinase gene in a liquid media comprising disodium tyrosine at a temperature greater than 25° C. and less than 37° C., and obtaining melanin from the culture. Optionally, the tyrosinase from Bacillus megaterium is expressed under the control of an inducible promoter.

In a further embodiment, the temperature is between 26° C. and about 35° C. In a still further embodiment, the temperature is 30° C.

In additional embodiments, the liquid media is VnM9v2 and the culture is grown in a shaker flask, or the liquid media is M9v3 and the culture is grown in a bioreactor.

### BRIEF DESCRIPTION OF THE DRAWINGS

FIG. 1 provides a table showing key developments towards improving melanin production yields. Significant changes include the switch from E. coli to V. natriegens at iteration 2, the change in media at iteration 3, the use of disodium tyrosine at iteration 4, the reduced temperate at iteration 5, and the further change in media at interation 6 (starred). Approximate respective yields obtained after each iteration is reported at the bottom. For the sixth iteration, a VnM9v3 media was developed that was optimized specifically for culture growth in bioreactors as compared to the VnM9v2 formulation that was optimized for shaker flask cultures.

FIG. 2 shows comparative results of various microbial processes for obtaining melanin, adapted from Choi, “Bioprocess of Microbial Melanin Production and Isolation,” Front. Bioeng. Biotechnol., 2021, DOI: 10.3389/fbioe.2021.765110. Here, melC is tyrosinase from Bacillus megaterium, cyp102G4 is cytochrome P450 monooxygenase from Streptomyces cattleya, 4-hppd is 4-hydroxyphenylpyruvate dioxygenase, and tyr1 is tyrosinase from Bacillus megaterium.

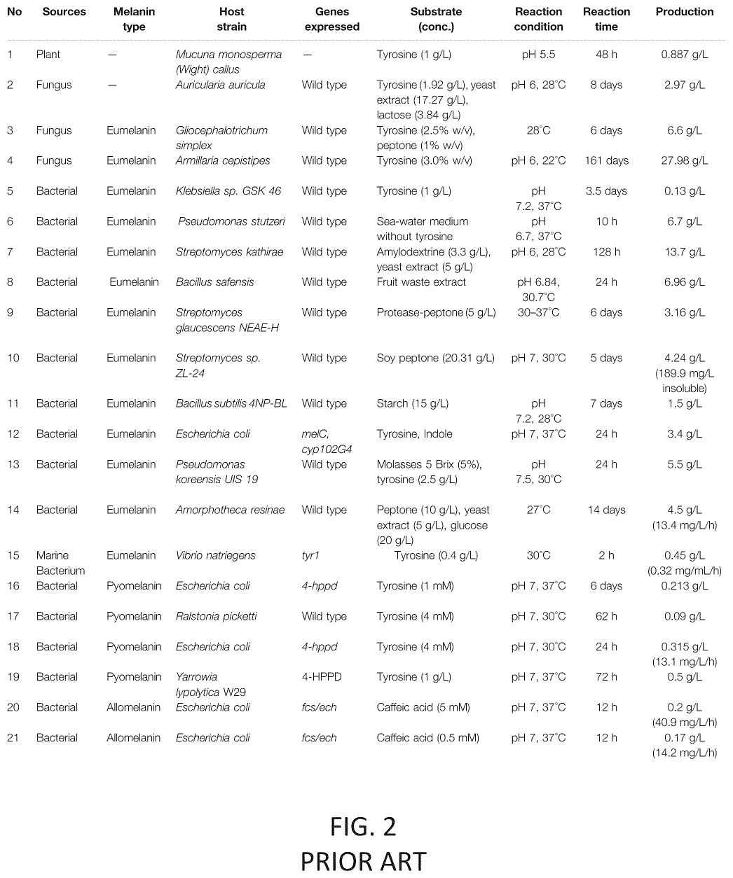

### DETAILED DESCRIPTION

### Definitions

Before describing the present invention in detail, it is to be understood that the terminology used in the specification is for the purpose of describing particular embodiments, and is not necessarily intended to be limiting. Although many methods, structures and materials similar, modified, or equivalent to those described herein can be used in the practice of the present invention without undue experimentation, the preferred methods, structures and materials are described herein. In describing and claiming the present invention, the following terminology will be used in accordance with the definitions set out below.

As used herein, the singular forms “a”, “an,” and “the” do not preclude plural referents, unless the content clearly dictates otherwise.

As used herein, the term “and/or” includes any and all combinations of one or more of the associated listed items.

As used herein, the term “about” when used in conjunction with a stated numerical value or range denotes somewhat more or somewhat less than the stated value or range, to within a range of ±10% of that stated.

### Overview

Compared to the process for obtaining melanin described Wang et al., the approach described herein obtained approximately eight times higher yield. Improvements arose from a combination of changes in growth media, culture temperature, and tyrosine source.

A defined minimal media was developed, optimized for V. natriegens growth, focusing on a number of factors including essential elemental sources/concentrations (nitrogen, sulfur, phosphorous), salt concentrations (NaCl), carbon sources and concentrations (such as glucose and glycerol as well as alternative carbon sources (xylose, citrate, lactose, etc.). Also examined were additional supplements (such as casamino acids, buffers, aspartate, thiamine, and metals). This work led to a media formulation termed “VnM9v2.”

A comparative analysis was made of enzymes for biosynthesis of melanin, comparing Tyr1 vs HpaBC vs MelA. Tyr1 was found to be the most efficient of these melanin biosynthetic enzymes.

Also examined were various promoters of expression, including constitutive and inducible promoters, the latter including those inducible by IPTG (isopropyl ß-D-1-thiogalactopyranoside), copper, and arabinose. While the IPTG promoter was found to produce the best results, another promoter could be used to avoid the cost of using IPTG.

Regarding substrates, the low solubility of substrate tyrosine in the growth medium (no more than ˜0.5 g/L) limited the yield of product melanin. Switching from tyrosine to disodium tyrosine as a substrate increased product yields, as disodium tyrosine has much greater solubility than tyrosine (greater than 100-fold) allowing for addition of much more substrate to increase the product yield.

Additional improvements were had by using an initial growth phase at 37° C. followed by incubation at 30° C. during biosynthesis.

The above-described VnM9v2 media, used for shaker flask cultures, was further refined in what was termed VnM9v3 specifically formulated for optimal growth in bioreactor. This involved two key changes. First was substitution of NaCl with Na₂SO₄. Although high sodium ion content is required for optimal V. natriegens growth, the high chloride ion content associated with use of NaCl would tend to cause bioreactor corrosion. This substitution mitigates this concern. The second change was a reduction in phosphate content from 100 mM to 20 mM final concentration. This presents a substantial cost savings for large scale production. Additionally, since bioreactors typically have automated pH sensing and control, the higher buffering capacity required to maintain pH in shake flasks cultures (100 mM phosphate) is not required in bioreactors in which a lower buffering capacity is preferred (20 mM phosphate).

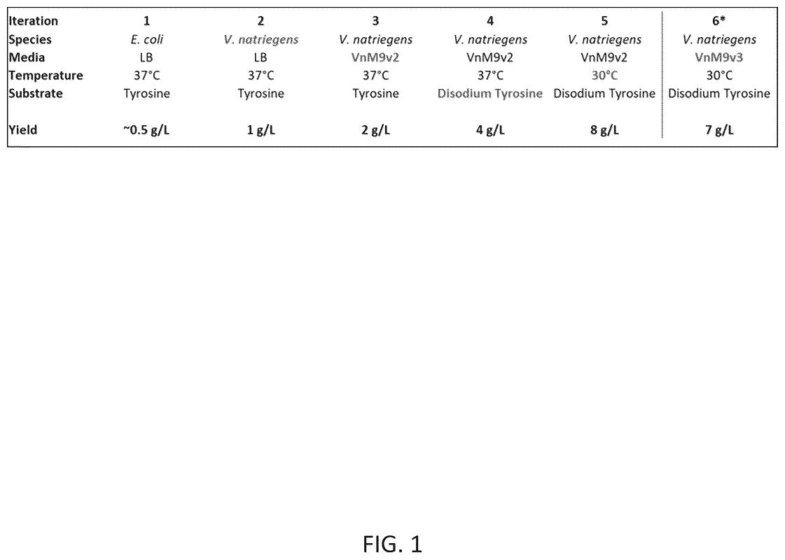

The combination of incubation at 30° C. during biosynthesis (instead of 37° C.) and the use of the optimized media surprisingly and unexpectedly resulted in a large increase in the melanin yield (FIG. 1). The resulting bioproduction process has yielded one of the highest yields and productivities currently reported in the literature, while using the convenient Vibrio natriegens platform.

### Examples

The stocks used in the V. natriegens high density growth minimal media for melanin biosynthesis termed VnM9v2, optimized for use in shaker flask culture, were as follows. All were autoclaved except as noted with an asterisk.
1. 20× Minimal Salts
   1.60 M K₂HPO₄
   0.4 M NaH₂PO₄
   1 M NH₄Cl
   0.1 M Na₂SO₄
2. 5 M NaCl
3. 1 M MgSO₄
4. 100 mM CaCl₂
5. 40× Carbon Sources
   20% (w/v) Glycerol
   20% (w/v) Glucose
6. 10% (w/v) Casamino Acids*
7. 25% (w/v) Aspartate
   84 mL water
   25 g Aspartic Acid
   8 g NaOH
8. 0.1 M FeCl₃*
   Dissolved in 0.1 M HCl
9. 1000× Essential Metals Mix*^{#}
   50 mM FeCl₃ (from 0.1 M stock in HCl)
   10 mM MnCl₂
   10 mM ZnSO₄
   2 mM CoCl₂
   2 mM CuCl₂
   2 mM NiCl₂
   2 mM Na₂MoO₄
   2 mM Na₂SeO₃
   2 mM H₃BO₃
10. 10 mM Thiamine HCl* * Do not autoclave. Filter sterilize with 0.2 μm filters.^{#}Make 0.1 M stocks of each element. Only FeCl₃ stock is dissolved in 0.1 M HCl

From these stocks, the recipe for preparing one liter of VnM9v2 media is as follows:
840 mL Autoclaved Water
50 mL 20× Minimal Salts
1 mL 1 M MgSO₄
3 mL 100 mM CaCl₂
20 mL 40× Carbon Sources
20 mL 10% (w/v) Casamino Acids
8 mL 25% (w/v) Aspartate
0.2 mL 1000× Essential Metals Mix
0.1 mL 10 mM Thiamine HCl
An appropriate concentration of selective antibiotic

This results in the following final concentrations in the VnM9v2 media ready for use:
80 mM K₂HPO₄
20 mM NaH₂PO₄
50 mM NH₄Cl
5 mM Na₂SO₄
275 mM NaCl
1 mM MgSO₄
0.3 mM CaCl₂
0.4% (wt/vol) Glycerol
0.4% (wt/vol) Glucose
0.2% (wt/vol) Casamino Acids
0.2% (wt/vol) Aspartate
0.2× Essential Trace Metals Mix
1 μM Thiamine

The optimized process, for cultures in shaker flasks, is as follows:
Inoculate 1 L of media to a starting OD₆₀₀=0.1 from a starter culture of V. natriegens transformed with pJV-Tyr1 (IPTG inducible Tyr1 plasmid)
Grow culture at 37° C., 200 rpm, until OD₆₀₀˜ 0.8 (roughly 2 hours)
Add IPTG to a final concentration of 1 mM
Reduce temperature to 30° C. and continue to incubate at 200 rpm for another 3 hours to allow for Tyr1 induction
After induction period, supplement cultures with 40 μM CuSO₄ and 8 mg/mL disodium tyrosine
Continue incubating cultures at 30° C., 200 rpm, overnight (minimum of 12 hours) for bioproduction of melanin

The stocks used in the V. natriegens high density growth minimal media for melanin biosynthesis termed VnM9v3, optimized for use in a bioreactor, were as follows. All were autoclaved except as noted with an asterisk.
1. 10× Minimal Salts
   160 mM K₂HPO₄
   40 mM NaH₂PO₄
   500 mM NH₄Cl
   1.3 M Na₂SO₄
2. 1 M MgSO₄
3. 100 mM CaCl₂
4. 40× Carbon Sources
   20% (w/v) Glycerol
   20% (w/v) Glucose
5. 10% (w/v) Casamino Acids*
6. 25% (w/v) Aspartate
   84 mL water
   25 g Aspartic Acid
   8 g NaOH
7. 0.1M FeCl₃*
   Dissolved in 0.1 M HCl
8. 1000× Essential Metals Mix*^{#}
   50 mM FeCl3 (from 0.1 M stock in HCl)
   10 mM MnCl₂
   10 mM ZnSO₄
   2 mM CoCl₂
   2 mM CuCl₂
   2 mM NiCl₂
   2 mM Na₂MoO₄
   2 mM Na₂SeO₃
   2 mM H₃BO₃
9. 10 mM Thiamine HCl* * Do not autoclave. Filter sterilize with 0.2 μm filters.^{#}Make 0.1 M stocks of each element. Only FeCl₃ stock is dissolved in 0.1 M HCl

From these stocks, the recipe for preparing one liter of VnM9v3 media is as follows:
845 mL Autoclaved Water
100 mL 10× Minimal Salts
1 mL 1 M MgSO₄
3 mL 100 mM CaCl₂
20 mL 40× Carbon Sources
20 mL 10% (w/v) Casamino Acids
8 mL 25% (w/v) Aspartate
0.2 mL 1000× Essential Metals Mix
0.1 mL 10 mM Thiamine HCl
An appropriate concentration of selective antibiotic

This results in the following final concentrations in the VnM9v3 media ready for use:
16 mM K₂HPO₄
4 mM NaH₂PO₄
50 mM NH₄Cl
130 mM Na₂SO₄
1 mM MgSO₄
0.3 mM CaCl₂
0.4% (wt/vol) Glycerol
0.4% (wt/vol) Glucose
0.2% (wt/vol) Casamino Acids
0.2% (wt/vol) Aspartate
0.2× Essential Trace Metals Mix
1 μM Thiamine

Melanin production in a bioreactor was accomplished as follows using an Eppendorf DASBox system with additional DASGIP MP8 pump controller module and DASGIP OD4 sensor module. Experimental parameters were all adjusted and set using the accompanying DASware control 5 software.

100 mL filter sterilized VnM9v3+30 μg/ml chloramphenicol was added to autoclaved reactor vessels. Overnight culture of V. natriegens transformed with pJV-Tyr1 was added to final OD₆₀₀ of 0.1. Cultivation of cells were carried out a temperature of 37° C. until OD₆₀₀ reached 1.0. Temperature was reduced to 30° C. and IPTG was added to a final concentration of 0.1 mM and incubated for an additional 3 h. After induction period, 40 μM CuSO₄ and 8 mg/mL disodium tyrosine were added to reactors.

The reactor parameters were:
Temperature was maintained at 30° C. after induction period (37° C. pre-induction) using a temperature probe.
Dissolved oxygen (DO) was set to 30% and was sensed by an O₂ sensor and controlled by an automated cascade consisting of increased impeller speeds and increased air flow:
   p-value=0.5
   N: 0-40%, 400-1200 rpm
   XO2: 40-80%, 21% constant
   F: 40-100%, 6-18 sL/h
pH set point was set at 7.0 and monitored using a pH probe
   Used 1M H₃PO₄ and 3M NaOH for automated pH control
Level sensor was used for automatic anti-foam injections using stock of 1% propylene glycol 2000 (PPG2000) and a custom level sensor script:
   1) If p.LvlPV>400
   2) p.FCSP=10
   3) Else
   4) p.FCSP=0
   5) End if

### Further Embodiments

In various aspects, the growth media can have a chloride ion concentration in the range of 25-75 mM while maintaining sufficient sodium for V. natriegens growth (typically by using a non-chloride salt as a sodium source to reach at least 100 mM sodium). In further aspects, the growth media can have a phosphate ion concentration in the range of 10-30 mM.

### CONCLUDING REMARKS

Although the present invention has been described in connection with preferred embodiments thereof, it will be appreciated by those skilled in the art that additions, deletions, modifications, and substitutions not specifically described may be made without departing from the spirit and scope of the invention. Terminology used herein should not be construed as being “means-plus-function” language unless the term “means” is expressly used in association therewith.

### REFERENCES

1. U.S. Pat. No. 11,162,212.
2. Wang et al., “Melanin Produced by the Fast-Growing Marine Bacterium Vibrio natriegens through Heterologous Biosynthesis: Characterization and Application,” Applied and Environmental Microbiology, 2020, 86 (5), e02749-19. DOI: 10.1128/AEM.02749-19
3. Choi, “Bioprocess of Microbial Melanin Production and Isolation,” Front. Bioeng. Biotechnol., 2021, DOI: 10.3389/fbioe.2021.765110

What is claimed is:

1. A method for producing melanin comprising:
incubating a culture of Vibrio natriegens expressing a heterologous tyrosinase in a liquid media comprising disodium tyrosine at a temperature greater than 25° C. and less than 37° C., and obtaining melanin therefrom.

2. The method of claim 1 wherein the temperature is between 26° C. and 35° C.

3. The method of claim 2 wherein the temperature is about 30° C.

4. The method of claim 1 wherein the liquid media is VnM9v2 or VnM9v3.

5. The method of claim 1, wherein the heterologous tyrosinase is Tyr1 from Bacillus megaterium and the expression is under the control of an inducible promoter.

6. The method of claim 1, further comprising first growing the culture at a temperature of 37° C. prior to inducing expression of said tyrosinase and reducing the temperature to said greater than 25° C. and less than 37° C.

7. The method of claim 1, wherein the growth media has a chloride ion concentration in the range of 25-75 mM and/or a phosphate ion concentration in the range of 10-30 mM.

8. A method for producing melanin comprising:
incubating in a liquid media a culture of Vibrio natriegens expressing a heterologous tyrosinase from Bacillus megaterium at a temperature between 26° C. and 35° C., inclusive, and obtaining melanin from the culture
wherein the liquid media comprises disodium tyrosine and the following components: 16 mM K₂HPO₄, 4 mM NaH₂PO₄, 50 mM NH₄Cl, 130 mM Na₂SO₄, 1 mM MgSO₄, 0.3 mM CaCl₂, 0.4% (wt/vol) Glycerol, 0.4% (wt/vol) glucose, 0.2% (wt/vol) casamino acids, 0.2% (wt/vol) aspartate, and 1 μM Thiamine, wherein each component is initially present in an amount within +/−20% from the listed quantity.

9. The method of claim 8 wherein the temperature is about 30° C.

10. The method of claim 8, further comprising first growing the culture at a temperature of 37° C. prior to inducing expression of said tyrosinase and reducing the temperature to said greater than 25° C. and less than 37° C.
